(19) United States
(12) Patent Application Publication
(10) Pub. No.: US 2024/0366657 A1
(43) Pub. Date: Nov. 7, 2024

(54) WATER-BASED IRON SUPPLEMENT FORMULATIONS FOR DOSING ANIMAL NEONATES

(71) Applicant: ALBERTA VETERINARY LABORATORIES LTD., Calgary (CA)

(72) Inventors: Merle Olson, Calgary (CA); Adam Olson, Calgary (CA); David Ireland, Calgary (CA)

(21) Appl. No.: 18/552,657

(22) PCT Filed: Mar. 25, 2022
(86) PCT No.: PCT/CA2022/050445

§ 371 Date: Sep. 26, 2023

Related U.S. Application Data
(60) Provisional application No. 63/166,430, filed on Mar. 26, 2021.

(51) Int. Cl. A61K 33/26; A61K 9/00; A61K 31/07; A61K 31/194; A61K 31/355; A61K 31/593; A61K 31/714; A61K 33/04; A61K 47/36; A61P 3/02

(52) U.S. Cl. CPC A61K 33/26; A61K 9/0056; A61K 31/07; A61K 31/194; A61K 31/355; A61K 31/593; A61K 31/714; A61K 33/04; A61K 47/36; A61P 3/02

(57) ABSTRACT

Disclosed are oral veterinary suspensions for delivering supplemental iron to a neonatal animal. The oral veterinary suspensions comprise water, an iron salt that is iron fumarate, iron sulfate, or a combination thereof suspended in the water, and a suspending agent. Also disclosed are methods of treating or preventing iron deficiencies to neonatal animals, as well as uses of the oral veterinary suspensions.

### CROSS-REFERENCE TO RELATED APPLICATIONS

This application is a U.S. national phase of and claims priority to International Application No. PCT/CA2022/050445 filed on Mar. 25, 2022, which claims priority to and benefit of U.S. Patent Application Ser. No. 63/166,430 filed on Mar. 26, 2021, which are hereby incorporated by reference in their entireties.

### TECHNICAL FIELD

This disclosure generally relates to nutritional supplements for neonatal animals. More specifically, this disclosure pertains to water-based suspensions for delivery of selected nutrients to neonatal animals.

### BACKGROUND

Iron is an important trace mineral in mammals. In general, iron is required for effective hematopoiesis as well as a number of intracellular and intercellular reactions. However, many animals such as dogs, cats, cows, pigs, lambs, foals, kids, and the like are iron deficient when they are born. In fact, in some animals, milk provided by their mothers is a poor source iron and so may not fully support the nutritional requirements of the neonatal animals. As result, neonatal animals are often at risk of becoming anemic.

Conventionally, iron supplements are administered parenterally via injection. In more detail, the injection is typically an intramuscular injection of iron dextran. However, the injections are often painful for the neonatal animals. In fact, in some cases, the animals may suffer from injection site infections after administration. Further, if the injections are not properly dosed, they may have toxic effects on the animals such as inducing hepcidin expression, which may actually reduce the bioavailability of the supplemental iron. As well, the injections are often more challenging for those administering the supplements, as it may be difficult to restrain the animals to correctly inject them. This problem is especially relevant to farmers, who in some cases must administer a large number of iron supplement injections over a short period of time.

One alternative to intramuscular injection is oral administration of iron supplements to animals. The oral supplements may be formulated as feed supplements or single-dose supplements. In feed supplements, iron dextran or iron sulfate may be added to an animal's food for voluntary intake. However, neonatal animals are sometimes reluctant to try food sources other than milk. As well, because their molecular absorption mechanisms are not fully developed, the iron is sometimes not completely absorbed when the animals do eat. On the other hand, if the neonatal animal eats too much of the feed, the supplemental iron may have adverse effects on the animal's gastrointestinal tract due to the oxidative toxicity of ferrous iron. As a result, feed supplements may not be suitable for consistent and accurate administration of supplemental iron.

Single-dose supplements are typically formulated as oil-based pastes. However, the pastes often do not provide sufficient amounts of iron to the animals due in part to their iron content as well as the difficulty associated with actually administering the pastes to the animals. For example, the pastes may be difficult to administer to animals due to their palatability and because their physical properties make them more difficult to for someone to force-feed to the animals. Further, because of the oil-based formulation, the pastes often destabilize and become rancid, further decreasing their palatability. As a result, single-dose oral iron supplements are generally ineffective at providing animals with the necessary amounts of supplemental iron.

Typical iron formulations have low concentrations of iron requiring large treatment volumes of iron suspensions to be administered orally. This dosing approach commonly results in aspiration and regurgitation of the administered iron treatments by the treated animals.

### SUMMARY

Embodiments of the present disclosure generally relate to oral veterinary suspensions. The suspensions may be administered to neonatal animals to treat or prevent iron deficiencies therein.

According to an example of an embodiment disclosed herein, an oral veterinary suspension for delivering supplemental iron to a neonatal animal comprises water as a base and an iron salt that is iron fumarate, an iron sulfate, or a combination thereof suspended in the water. The suspension also comprises a suspending agent.

The present disclosure also relates to methods of treating or preventing an iron deficiency of a neonatal animal. In one example of an embodiment disclosed herein, the methods may comprise administering to the neonatal animal a selected volume of the oral veterinary suspension described herein.

The present disclosure also relates to uses of the oral veterinary suspension described herein for the treatment of prevention of an iron deficiency of an animal.

Other aspects and features of the present disclosure will become apparent to those ordinarily skilled in the art upon review of the following description of specific embodiments.

### BRIEF DESCRIPTION OF THE DRAWINGS

Further advantages, features, permutations and combinations of the present disclosure will now appear from the above and from the following detailed description of the various particular embodiments, taken together with the accompanying drawings appended herewith, each of which are intended to be non-limiting, in which:

FIG. 1 is a graph showing the average change in hemoglobin concentration for Oral-Fe1 treated calves compared to control calves by population and as seen between female and male animals.

FIG. 2 is a graph showing the average change in packed cell volume for Oral Fe1 treated calves compared to control calves by population and as seen between female and male animals.

### DETAILED DESCRIPTION

The embodiments of the present disclosure generally relate to oral veterinary suspensions, to methods of treating or preventing iron deficiencies in neonatal animals by administering the oral veterinary suspensions, and to uses of the oral veterinary suspensions for the treatment or prevention of iron deficiencies in neonatal animals.

In one embodiment of the present disclosure, there is provided an oral veterinary suspension for delivering supplemental iron to a neonatal animal. The oral veterinary suspension comprises water and an iron salt that is iron fumarate, an iron sulfate, or a combination thereof suspended in the water. In an embodiment, the iron salt is iron fumarate (e.g. ferrous fumarate). The oral veterinary suspension also comprises a suspending agent.

As used herein, “oral veterinary suspension” refers to a suspension for oral administration that may provide a one or more supplemental nutrients, vitamins, nutritional minerals, or the like to an animal. The suspensions of the present disclosure are water-based in that the components thereof are suspended in water.

As used herein, “neonatal animals” encompass those that are newly born to an age of about one month. Examples of animals that are suitable to receive the oral veterinary suspensions of the present disclosure include dogs, cats, pigs, cattle, horses, sheep, goats, and the like that are deficient in iron or that may be at risk of becoming iron deficient.

As used herein, “single-dose supplements” refer to those configured to deliver a selected amount of a supplement to an animal per each individual use. Single-dose supplements differ from those provided to an animal via voluntary intake in that a farmer or veterinarian may select the size of the dose and when it is to be administered, and then administer it all at once. It is to be noted that single-dose supplements may be administered to an animal more than once if need be.

According to some embodiments, the iron salt of the oral veterinary suspensions of the present disclosure is iron fumarate (also referred to as “ferrous fumarate”). In an embodiment, the iron fumarate may be present in the suspension at a concentration of about 100 mg/mL to about 400 mg/mL. In some aspects, the iron fumarate may be present at a concentration of about 200 mg/mL. In some aspects, the iron fumarate may be present at a concentration of about 300 mg/ml. In another embodiment, the iron salt of the oral veterinary suspensions may be iron sulfate. The iron sulfate may be included in the same concentrations as described above in relation to the iron fumarate. If both iron fumarate and iron sulfate are used, the total concentration of iron salt may be the same as the ranges outlined above. It is noted that the mg/mL refers to the mg of iron salt (or other component) per ml of water of the suspension.

Further, in an embodiment, the iron salt of the oral veterinary suspensions may have a particle size of about 25 μm to about 200 μm. In some aspects, the iron salt has a particle size of about 150 μm.

In some embodiments, the suspending agent of the oral veterinary suspensions may comprise xanthan gum, cellulose, nitrocellulose, the like, or a combination thereof. As will be appreciated, suspending agents are components of the suspension that help to reduce the rate at which other components of the suspension settle. In one aspect, the suspending agent may be xanthan gum. In some aspects, the suspending agent is present at a concentration of about 1 mg/mL to about 10 mg/mL. In a particular aspect, the suspending agent may be present at a concentration of about 5 mg/mL.

In a further embodiment, the oral veterinary suspensions may comprise a flavorant. The flavorant may be included to increase the palatability of the suspensions. In some aspects, the flavorant may be a sweetener. The sweetener may be a natural sweetener, an artificial sweetener, or a combination thereof. For example, the sweetener may be sucrose, stevia, erythritol, xylitol, sucralose, saccharin, aspartame, or any combination thereof. In a further aspect, the flavorant may be present in the suspension at a concentration of about 25 mg/mL to about 100 mg/mL.

In a yet further embodiment, the oral veterinary suspension may comprise one or more preservatives. The preservatives may be included to prevent or reduce the likelihood of microbial growth in the suspension to thereby further increase the shelf life thereof. The one or more preservatives may comprise a sorbate, sorbic acid, an ascorbate, ascorbic acid, or any combination thereof. In a further aspect, the one or more preservatives may comprise potassium sorbate. The one or more preservatives may each be included at a concentration of about 0.1 mg/ml to about 0.5 mg/mL.

In a further embodiment, the oral veterinary suspensions may comprise one or more vitamins and/or nutritional minerals. The one or more vitamins may comprise, for example and without limitation, vitamin A, vitamin E, vitamin B12, vitamin D3, or any combination thereof. In an embodiment, the one or more vitamins is all of vitamin A, vitamin E, vitamin B12, vitamin D3. In an embodiment, the one or more vitamins is vitamin A acetate, vitamin B12, vitamin E acetate, the like, or any combination thereof. The one or more nutritional minerals may comprise, for example and without limitation, sodium selenite.

The vitamins and/or nutritional minerals may be included in the oral veterinary suspensions at any suitable concentration. In an embodiment, the one or more vitamins and/or nutritional minerals may be present in the suspension at a concentration of about 0.01 mg/mL to about 100 mg/mL, more particularly about 0.01 mg/ml to about 50 mg/mL. For example and without limitation, if present, the vitamin A (e.g. vitamin A acetate) may be present at a concentration of about 25 mg/mL to about 50 mg/mL, vitamin E (e.g. vitamin E acetate) may be present at a concentration of about 25 mg/mL to about 50 mg/mL; vitamin B12 may be present at a concentration of about 0.1 mg/ml to about 10 mg/mL; and vitamin D3 may be present at a concentration of about 0.01 mg/ml to about 1.0 mg/mL. In another example, if present, the vitamin A acetate may be present at a concentration of about 35 mg/mL, the vitamin B12 may be present at a concentration of about 0.5 mg/mL, the vitamin E acetate may be present at a concentration of about 75 mg/mL, and the sodium selenite may be present at a concentration of about 0.05 mg/mL. In another example, if present, the vitamin A may be present at a concentration of about 50 mg/mL, the vitamin B12 may be present at a concentration of about 0.5 mg/ml, the vitamin E may be present at a concentration of about 30 mg/mL, the vitamin D3 may be present at a concentration of about 0.03 mg/mL, and the sodium selenite may be present at a concentration of about 0.5 mg/mL. Of course, other concentrations of vitamins and nutritional nutrients may be used if so desired.

In an embodiment, the oral veterinary suspension of the present disclosure comprise the following active and inactive ingredients as shown in Table 1:

TABLE 1
Exemplary Active and Inactive ingredients of an Oral Veterinary
Suspension of the Present Disclosure (Oral-Fe1)

| INGREDIENT | AMOUNT | |
|---|---|---|
| Ferrous Fumarate | 300 | mg/mL |
| Vitamin A | 50 | mg/mL |
| Vitamin E | 30 | mg/mL |
| Sodium selenite | 0.5 | mg/mL |
| Vitamin B12 | 0.5 | mg/mL |
| Vitamin D3 | 0.03 | mg/mL |

The oral veterinary suspensions of the present disclosure may be manufactured with relative ease. For example, according to one embodiment, a method of manufacturing an oral veterinary suspension of the present disclosure may comprise adding water to a mixing vessel, adding the suspending agent to the water to form a mixture, allowing the mixture to homogenize for about 5 min to about 30 min, adding the iron salt to the homogenized mixture to form a suspension, and allowing the suspension to further homogenize for about 15 min to about 45 min. If one or more additives such as vitamins, nutritional minerals, preservatives, flavorants, and the like are to be included in the suspension, they may be added to the water prior to the addition of the suspending agent and subsequently mixed therewith for about 10 min to about 40 min.

Once the oral veterinary suspension is produced, it may then be stored in any suitable storage container. For example, the oral veterinary suspension may be stored in bottles having a volume of 150 mL, 250 mL, 500 mL, or more or less if so desired, for future use. Alternatively, the oral veterinary suspensions may be stored in syringes for subsequent sale as pre-loaded syringes.

Another embodiment of the present disclosure relates to methods of treating or preventing an iron deficiency in a neonatal animal. The methods may comprise administering to the neonatal animal a selected volume of the oral veterinary suspension of the present disclosure.

According to an aspect, the oral veterinary suspension may be administered using a commercial doser such as those available from MAI Animal Health, Simcro Datamars, and other veterinary products suppliers. In such aspects, the dosers may be pre-loaded a volume of about to 50 mL to about to about 1 L of the suspension and configured to a selected volume of about 3 mL to about 20 ml of the oral veterinary suspension to the neonatal animal. In one aspect, administering the oral veterinary suspension to the neonatal animal may comprise administering a selected volume of about 1 mL, about 2 mL, about 3 mL, about 4 mL, about 5 mL, about 6 mL, about 7 mL, about 8mL, about 9 mL, about 10 mL, or more of the suspension to the neonatal animal. Alternatively, in another aspect, the oral veterinary suspensions may be administered via a syringe or a dosing gun to deliver the selected volumes specified above.

In an embodiment, administering the oral veterinary suspension to the neonatal animal may comprise administering a dose volume of about 3 mL, whereby the oral suspension comprises about 300 mg/ml of ferrous fumarate. In an embodiment, the neonate animal is a calve or a lamb. In an embodiment, the neonate animal is a lamb.

In an embodiment, administering the oral veterinary suspension to the neonatal animal may comprise administering a dose volume of about 10 mL, whereby the oral suspension comprises about 300 mg/ml of ferrous fumarate. In an embodiment, the neonate animal is a calve or a lamb. In an embodiment, the neonate animal is a calve.

Thus, another embodiment of the present disclosure relates to the use of the oral veterinary suspensions described herein for the treatment or prevention of an iron deficiency in a neonatal animal.

The oral veterinary suspensions disclosed herein may be easier for someone to administer to an animal than intramuscular injections and conventional single-dose oral iron supplements. As previously described herein, animals often have to be restrained to receive the injection and, if not done properly, may receive the injection to a part of their body not particularly suitable therefor. As well, as also described above, conventional single-dose oral iron supplements are generally formed as oil based pastes which may not be palatable to the animals and, because of the physical properties of the paste, may be difficult for a person to administer the dose to an animal.

Further, the oral veterinary suspensions of the present disclosure may avoid the potential adverse health effects that may be caused by conventional iron supplements. For example, intramuscular injections of iron dextran are painful and may lead to complications such as injection site infections, induced hepcidin expression, iatrogenic disease transmission, formation of abscesses at the injection site, and acute toxicity. As well, conventional formulations for oral administration such as feed supplements, if over-consumed by an animal, may be toxic to the animal's gastrointestinal tract.

Furthermore, the oral veterinary suspensions of the present disclosure may have an increased shelf-life compared to conventional single-dose oral supplements. For example, the suspensions of the present disclosure may be stable for at least a year. As well, if the iron supplement settles out of the suspension disclosed herein, the contents may simply be re-suspended, thereby allowing the suspension to be used effectively or stored for later use. In contrast, conventional single-dose iron supplements are oil-based that, when destabilized, become rancid and cannot be returned to their original form, thereby rendering the supplement useless. The single dosage oil-based iron supplements generally destabilize in less than one year.

### EXAMPLES

### Example 1

The aim of this study was to measure the hemoglobin, packed cell volume, and plasma ferritin in newborn lambs and the response of these variables to oral iron supplementation by a suspension of the present disclosure, at birth and 2 weeks later.

Forty-two (42) animals were enrolled during a spring lambing season. From April to May all multiple birth lambings had two of the animals enrolled within 0 to 2 days following birth. Animals entered the study when they were tagged, with even lambs receiving treatment with an oral iron suspension of the present disclosure and odd numbered lambs receiving a control saline treatment. After animals had their weight taken, a blood sample was collected and used for analysis of packed cell volume (PCV) and hemoglobin concentration. Afterward, plasma was then isolated and frozen for future ferritin analysis.

The oral veterinary suspension of supplemental iron (“Oral-Fe1”) comprised the following active and inactive ingredients as shown in Table 2:

TABLE 2
Oral-Fe1 - Active and Inactive ingredients

| INGREDIENT | AMOUNT | |
|---|---|---|
| Ferrous Fumarate | 300 | mg/mL |
| Vitamin A | 50 | mg/mL |
| Vitamin E | 30 | mg/mL |
| Sodium selenite | 0.5 | mg/mL |
| Vitamin B12 | 0.5 | mg/mL |
| Vitamin D3 | 0.03 | mg/mL |

The dose of Oral-Fe1 that was provided to the animals was 3.0 mL per lamb on days 0 to 3.

### Experimental Design

Twin lambs were selected from a commercial sheep operation. Animals were recruited from a ranch in Sothern Alberta. The sheep herd was around 350 ewes. Lambs are of Dorset cross ewes bred to Suffolk rams. The animals were ranch raised on well water, fed alfalfa hay, barley with UFA mineral supplements. Forty-two (42) animals were enrolled for this study. A summary of the sex breakdown for each group (Table 3) and the average weight difference of the enrolled animals by sex and treatment groups (Table 4) was recorded. More detailed information regarding enrolled animals was also recorded (data not shown).

TABLE 3
Count of Treated/Control by Sex

| | F | M | Total |
|---|---|---|---|
| Treated | 12 | 9 | 21 |
| Control | 11 | 10 | 21 |
| Total | 23 | 19 | 42 |

TABLE 4
Average weight difference in Kilograms of newborn
female and male lambs after treatment with Oral-
Fe1 (Treated) or Saline (Control) after 21 days.

| | Female | Male | Average |
|---|---|---|---|
| Treated | 3.62 | 4.09 | 3.83 |
| Control | 3.36 | 3.92 | 3.59 |
| Average | 3.49 | 4.01 | 3.72 |

Animals were enrolled in the study 0 to 2 days after birth (Table 5). Animals entered the study when they were tagged, with one lamb being treated with Oral-Fe1 and the other with a control treatment of saline. A time 0 blood sample was collected from the jugular vein (approximately 3 cc) in an EDTA coated tube. Treated twin animals (even tags) received 3 mL of Oral-Fe1 orally following tagging. Control animals (odd tags) received 10 ml of saline orally following tagging (Table 6). A final blood sample was collected in an EDTA coated tube 10 to 21 days following treatment (average was 18 days).

TABLE 5
Schedule of Events

| | | | | | | Health |
|---|---|---|---|---|---|---|
| | | | | | Blood | Assessment |
| Study | | | Eligibility | Treat- | Collec- | Clinical |
| Day | Birth | Weight | Allocation | ments¹ | tion | Recording |
| Day 0 | ● | ● | | | | |
| Day 0-2 | | | ● | ● | ● | ● |
| Day 14-21 | | | | | ● | ● |

¹3 mL Oral-Fe1 to even tag lambs, 10 mL saline to control odd tag lambs.

TABLE 6
Treatment Regime

| | No. | TREATMENT | | | | |
|---|---|---|---|---|---|---|
| Group | animals | Type | Route | Dosage | | Regimen |
| Treated | 21 | Oral-Fe1 | Oral | 3 | mL | Once |
| Control | 21 | Saline | Oral | 10 | mL | Once |

Analysis of Samples: Blood samples were collected from the jugular vein (approximately 3 cc) before treatment (Time 0) and between 10 and 23 days after treatment in an EDTA coated tube. Fresh blood samples were analyzed for PCV and hemoglobin. Following PCV and hemoglobin analysis, blood samples were then centrifuged at 2500 RPM for 7 minutes, and divided and stored in labeled screw top microcentrifuge tubes and frozen immediately at minus eighty (−80) degrees Celsius.

For packed cell volume (PCV) analysis: This was performed on fresh samples. For each sample, the PCV is the percentage of red blood cells (RBCs) per volume of blood and was determined by centrifugation, using the micro-hematocrit method and reported as percentage values.

For hemoglobin: This was performed on fresh whole blood. A colorimetric hemoglobin assay kit (Bioassay Systems; DIHB-250) was used. This kit is based on a Triton/NaOH method, in which the hemoglobin is converted into a uniform-colored end product. The intensity of color, measured at 400 nm, is directly proportional to hemoglobin concentration in the sample.

For Ferritin: This will be performed on frozen plasma samples at a later date. Samples will be batched. A ferritin assay kit will be used.

Results: Forty-two (42) animals were enrolled in the study, 21 of these animals received Oral-Fe1 treatment (treated group; T) and 21 received saline (control group; C). Animals were, on average, in the study for 18.2 days. Hemoglobin concentrations (g/L) and packed cell volumes (percent) were determined on whole blood samples collected before their initial treatment 0-2 days after birth, as well as on May 12th (i.e. between 10 and 23 days after treatment). Weights were collected at these processing times. The average hemoglobin concentration for the control and treated animals can be seen in Table 7. The average packed cell volume (as a percent) is presented in Table 8. Hemoglobin concentration at birth for each treatment group is presented in Table 9, where animals with a concentration <125 g/L were categorized as anemic, and those above as healthy. On May 12, the anemia status for each group is presented in Table 10. The change in health as a function of the change of hemoglobin concentration for each treatment group is presented as “improved”, representing increased hemoglobin concentration of more than 5 g/L, “same” representing change was between −5 and 5 g/L, or “decline” representing a decrease of more than 5 g/L (Table 11). Table 12 shows the weight change for each treatment group from initial birth to May 12.

TABLE 7
Average hemoglobin concentration values for
control and Oral-Fe1 treated newborn lambs

| | Average | | | Average | | |
|---|---|---|---|---|---|---|
| | Day 0 | | | May 12 | | |
| | Conc | | n | Conc | | n |
| | (g/L) | StdDev | (Count) | (g/L) | StdDev | (Count) |
| Control | 128.44 | 19.47 | 21 | 123.73 | 9.99 | 19 |
| Treated | 129.36 | 27.14 | 21 | 128.67 | 8.77 | 18 |
| Population | 128.90 | 23.33 | 42 | 126.13 | 9.62 | 37 |

TABLE 8
Average packed cell volume (PCV as a percent) for control
(saline) and treated (Oral-Fe1) newborn lambs

| | Average | | | Average | | |
|---|---|---|---|---|---|---|
| | Day 0 | | | May 12 | | |
| | PCV % | StdDev | n (Count) | PCV % | StdDev | n (Count) |
| Control | 39.10 | 4.53 | 10 | 35.63 | 3.13 | 19 |
| Treated | 39.50 | 6.29 | 10 | 35.94 | 2.86 | 18 |
| Total | 39.30 | 5.34 | 20 | 35.78 | 2.96 | 37 |

TABLE 9
Count of hemoglobin status at birth. Anemic (hemoglobin
<125 g/L) or healthy (hemoglobin >125 g/L)

| | Anemic | Healthy | Total |
|---|---|---|---|
| Control | 10 | 11 | 21 |
| Treated | 9 | 12 | 21 |
| Total | 19 | 23 | 42 |

TABLE 10
Count of hemoglobin status on second blood sample (May 12). Anemic
(hemoglobin <125 g/L) or healthy (hemoglobin >125 g/L)

| | Anemic | Healthy | Total |
|---|---|---|---|
| Control | 10 | 9 | 19 |
| Treated | 5 | 13 | 18 |
| Total | 15 | 22 | 37 |

TABLE 11
Difference between hemoglobin values (g/L) on May 12 and Day 0

| | Decline¹ | Improved² | Same³ | Total |
|---|---|---|---|---|
| Control | 8 | 8 | 3 | 19 |
| Treated | 7 | 8 | 3 | 18 |
| Total | 15 | 16 | 6 | 37 |

¹“decline” more than 5 g/L decrease
²“improved” by more than 5 g/L
³“same” change was between −5 and 5 g/L

TABLE 12
Weight change from birth to the second weight (May 12)

| | Average Birth Wt | Average Day May 12 Wt | Average Wt Change |
|---|---|---|---|
| Control | 5.50 +/− 1.07 | 9.02 +/− 2.17 | 3.59 +/− 1.68 |
| Treated | 5.31 +/− 1.12 | 9.19 +/− 1.92 | 3.83 +/− 1.39 |
| P value | 0.39 | 0.94 | 0.47 |
| (Paired T test) | | | |

An anemic cut off value of 125 g/L of hemoglobin was chosen. At this cut off, 43% of treated and 47% control animals were anemic at birth. At time of second sampling, 28% of the treated animals were anemic, while 52% of control animals were anemic. There was no significant difference in birth and second sampling hemoglobin concentration between control and treated animals. There was a trend for treated animals to have a larger increase in hemoglobin concentration.

There was no significant difference in birth weight, second sample weight, or weight change between treatment and control animals (Table 12). There was a trend for treated animals to have a greater increase in weight gain (3.83 vs 3.59 kg).

### Example 2

The aim of this study was to measure the hemoglobin, packed cell volume, and plasma ferritin in newborn calves and the response of these variables to oral iron supplementation by a suspension of the present disclosure, at birth and 2 weeks later.

Forty-four (44) animals were enrolled during a spring calving season. From April to May newborns calves were enrolled within 0 to 2 days following birth. Animals entered the study when they were tagged, with half receiving treatment with an oral iron suspension of the present disclosure and the other half receiving a control saline treatment, in an alternating sequence. After animals had their tag, a blood sample was collected and used for analysis of packed cell volume (PCV) and hemoglobin concentration. Afterward, plasma was then isolated and frozen for future ferritin analysis.

The oral veterinary suspension of supplemental iron (“Oral-Fe1”) comprised the following active and inactive ingredients as shown in Table 13:

TABLE 13
Oral-Fe1 - Active and Inactive ingredients

| INGREDIENT | AMOUNT | |
|---|---|---|
| Ferrous Fumarate | 300 | mg/mL |
| Vitamin A | 50 | mg/mL |
| Vitamin E | 30 | mg/mL |
| Sodium selenite | 0.5 | mg/mL |
| Vitamin B12 | 0.5 | mg/mL |
| Vitamin D3 | 0.03 | mg/mL |

The dose of Oral-Fe1 that was provided to the animals was 10.0 mL per lamb on day 0.

### Experimental Design

Calves were selected from a commercial cow/calf operation in Sothern Alberta. The animals were ranch raised on well water, on free-range grazing supplemented with bales weekly. Forty-four (44) animals were enrolled for this study. A summary of the sex breakdown for each group (Table 14) was recorded and the average estimated enrollment weight is presented in Table 15. More detailed information regarding the enrolled animals was also recorded (data not shown).

TABLE 14
Count of Treated/Control by Sex

| | F | M | Total |
|---|---|---|---|
| Treated | 11 | 12 | 23 |
| Control | 7 | 14 | 21 |
| Total | 18 | 26 | 44 |

TABLE 15
Average estimated weight in pounds of newborn
female and male calves before study treatments.

| | Female | Male | Average |
|---|---|---|---|
| Treated | 81.36 | 86.67 | 84.13 |
| Control | 79.29 | 87.50 | 84.76 |
| Average | 80.56 | 87.12 | 84.43 |

Animals entered the trial 0 to 2 days after birth (Table 16). They entered the study when they are tagged. An estimated body weight was recorded to ensure that the treatment groups had comparable start animals. A time 0 blood sample was collected from the jugular or coccygeal vein (approximately 3 cc) in an EDTA tube. Treated animals received 10 mL of treatment following tagging and control animals received 10 ml of saline following tagging (Table 17). Treatment and control animals were determined based on sequence of birth starting with a treatment calf and alternating until 44 animals were enrolled into the study. A final blood sample was collected in an EDTA tube blood on May 30 when all the animals were processed. Average age was (32.4 days).

TABLE 16
Schedule of events

| | | Estimated | Eligi- | | | Health |
|---|---|---|---|---|---|---|
| | | Body | bility | | Blood | Assessment |
| Study | | Weight | Allo- | Treat- | Collec- | Clinical |
| Day | Birth | (lbs) | cation | ments¹ | tion | Recording |
| Day 0 | ● | | | | | |
| Day 0-1 | | ● | ● | ● | ● | ● |
| Day 14-43 | | | | | ● | ● |

TABLE 17
Treatment Regime

| | No. | TREATMENT | | | |
|---|---|---|---|---|---|
| Group | animals | Type | Route | Dosage | Regimen |
| Treated | 23 | Oral-Fe1 | Oral | 10 mL | Once |
| Control | 21 | Saline | Oral | 10 mL | Once |

Analysis of Samples: Blood samples were collected from the jugular vein (approximately 3 cc) before treatment (Time 0) and between 13 and 43 days after treatment in an EDTA coated tube. Fresh blood samples were analyzed for PCV and hemoglobin. Following PCV and hemoglobin analysis, blood samples were then centrifuged at 2500 RPM for 7 minutes, and divided and stored in labeled screw top microcentrifuge tubes and frozen immediately at minus eighty (−80) degrees Celsius.

For packed cell volume (PCV) analysis: This was performed on fresh samples. For each sample, the blood PCV is the percentage of red blood cells (RBCs) per volume of blood and was determined by centrifugation, using the micro-hematocrit method and reported as percentage values.

For hemoglobin: This was performed on fresh whole blood. A colorimetric hemoglobin assay kit (Bioassay Systems; DIHB-250) was used. This kit is based on a Triton/NaOH method, in which the hemoglobin is converted into a uniform-colored end product. The intensity of color, measured at 400 nm, is directly proportional to hemoglobin concentration (g/L) in the sample.

For Ferritin: This will be performed on frozen plasma samples at a later date. Samples will be batched. A ferritin assay kit will be used.

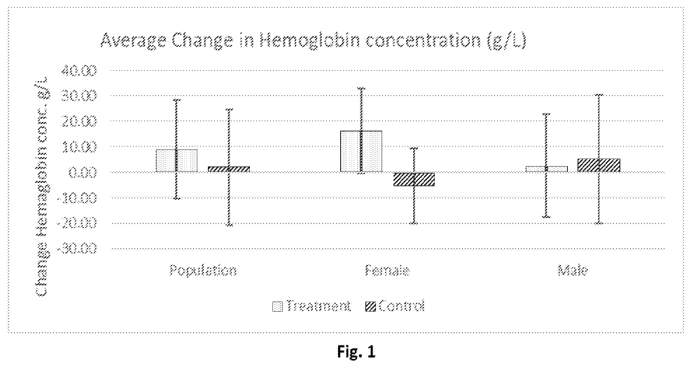

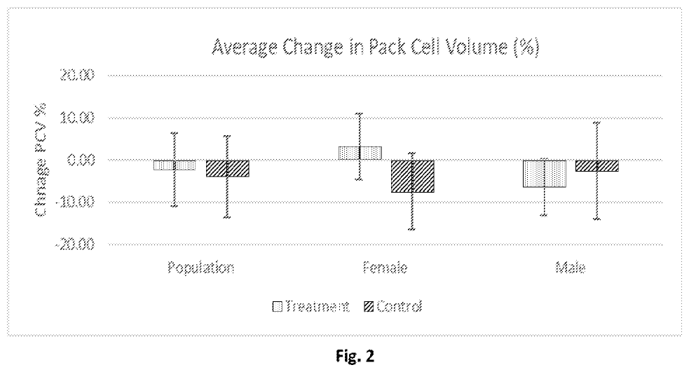

Results: Forty-four (44) animals were enrolled in the study, 23 of these animals received Oral-Fe1 treatment (treated group) and 21 received saline (control group). Animals were, on average, in the study for 32.4 days. Hemoglobin concentrations (g/L) and packed cell volumes (percent) were determined on whole blood samples collected before their initial treatment 0-1 day after birth, as well as on May 30th. The average hemoglobin concentration for the control and treated animals can be seen in Table 18. The average packed cell volume (as a percent) is presented in Table 19. The change in hemoglobin concentration from birth for each treatment group and separated by sex is presented in FIG. 1. FIG. 2 presents the change in packed cell volume for treated calves compared to control and separated by sex. The average change differences of hemoglobin concentration and packed cell volume between treatment groups was compared and a non-significant trend was found (Table 20).

TABLE 18
Average hemoglobin concentration values for
control and Oral-Fe1 treated newborn calves.

| | Average | | | Average | | |
|---|---|---|---|---|---|---|
| | Day 0 | | | May 30 | | |
| | Conc | | n | Conc | | n |
| | (g/L) | StdDev | (Count) | (g/L) | StdDev | (Count) |
| Treated | 113.74 | 19.26 | 23 | 122.82 | 10.64 | 23 |
| Control | 116.35 | 18.55 | 21 | 118.77 | 12.80 | 19 |
| Population | 114.99 | 18.75 | 44 | 120.99 | 11.70 | 42 |

TABLE 19
Average packed cell volume (PCV as a percent) for control
(saline) and treated (Oral-Fe1) newborn calves

| | Average | | | Average | | |
|---|---|---|---|---|---|---|
| | Day 0 | | | May 30 | | |
| | PCV % | StdDev | n (Count) | PCV % | StdDev | n (Count) |
| Treated | 39.31 | 9.67 | 16 | 37.61 | 3.13 | 23 |
| Control | 39.62 | 8.44 | 13 | 36.26 | 4.09 | 19 |
| Population | 39.45 | 8.98 | 29 | 37.00 | 3.62 | 42 |

TABLE 20
Change of Hemoglobin Concentration (Hb) and packed cell
volume (PCV) between May 30 blood sample from Day 0
(birth) blood sample for Oral-Fe1 treated animals compared
to control animals. Single tailed t-test between treatment
and control values was not significant.

| | Average of | Average of |
|---|---|---|
| | Change Hb | Change |
| | (g/L) | PCV % |
| Treatment | 9.08 | −2.25 |
| Control | 1.95 | −4.08 |
| Population | 5.85 | −3.07 |
| T test | 0.1380 | 0.2974 |

There was a trend for treated animals to have a larger increase in hemoglobin concentration. Weight was not precisely measured in this study in the follow up processing and only estimated at treatment to ensure even treatment groups.

Overall, there appears to be a benefit in providing an iron/vitamin supplement to calves.

In the present disclosure, all terms referred to in singular form are meant to encompass plural forms of the same. Likewise, all terms referred to in plural form are meant to encompass singular forms of the same. Unless defined otherwise, all technical and scientific terms used herein have the same meaning as commonly understood by one of ordinary skill in the art to which this disclosure pertains.

As used herein, the term “about” refers to an approximately +/−10% variation from a given value. It is to be understood that such a variation is always included in any given value provided herein, whether or not it is specifically referred to.

It should be understood that the compositions and methods are described in terms of “comprising,” “containing,” or “including” various components or steps, the compositions and methods can also “consist essentially of or “consist of the various components and steps. Moreover, the indefinite articles “a” or “an,” as used in the claims, are defined herein to mean one or more than one of the element that it introduces.

For the sake of brevity, only certain ranges are explicitly disclosed herein. However, ranges from any lower limit may be combined with any upper limit to recite a range not explicitly recited, as well as, ranges from any lower limit may be combined with any other lower limit to recite a range not explicitly recited, in the same way, ranges from any upper limit may be combined with any other upper limit to recite a range not explicitly recited. Additionally, whenever a numerical range with a lower limit and an upper limit is disclosed, any number and any included range falling within the range are specifically disclosed. In particular, every range of values (of the form, “from about a to about b,” or, equivalently, “from approximately a to b,” or, equivalently, “from approximately a-b”) disclosed herein is to be understood to set forth every number and range encompassed within the broader range of values even if not explicitly recited. Thus, every point or individual value may serve as its own lower or upper limit combined with any other point or individual value or any other lower or upper limit, to recite a range not explicitly recited.

Therefore, the present disclosure is well adapted to attain the ends and advantages mentioned as well as those that are inherent therein. The particular embodiments disclosed above are illustrative only, as the present disclosure may be modified and practiced in different but equivalent manners apparent to those skilled in the art having the benefit of the teachings herein. Although individual embodiments are discussed, the disclosure covers all combinations of all those embodiments. Furthermore, no limitations are intended to the details of construction or design herein shown, other than as described in the claims below. Also, the terms in the claims have their plain, ordinary meaning unless otherwise explicitly and clearly defined by the patentee. It is therefore evident that the particular illustrative embodiments disclosed above may be altered or modified and all such variations are considered within the scope and spirit of the present disclosure. If there is any conflict in the usages of a word or term in this specification and one or more patent(s) or other documents that may be incorporated herein by reference, the definitions that are consistent with this specification should be adopted.

Many obvious variations of the embodiments set out herein will suggest themselves to those skilled in the art in light of the present disclosure. Such obvious variations are within the full intended scope of the appended claims.

1. An oral veterinary suspension for delivering supplemental iron to a neonatal animal, the oral veterinary suspension comprising:
water;
an iron salt that is iron fumarate, iron sulfate, or a combination thereof suspended in the water; and
a suspending agent.

2. The oral veterinary suspension of claim 1, wherein the iron salt is iron fumarate.

3. The oral veterinary suspension of claim 1, comprising iron fumarate in an amount of about 100 mg/mL to about 400 mg/mL.

4. The oral veterinary suspension of claim 3, comprising iron fumarate in an amount of about 200 mg/mL.

5. The oral veterinary suspension of claim 1, wherein the suspending agent comprises xanthan gum, cellulose, nitrocellulose, or a combination thereof.

6. The oral veterinary suspension of claim 5, wherein the suspending agent is xanthan gum.

7. The oral veterinary suspension of claim 1, wherein the suspending agent is present in an amount of about 1 mg/ml to about 10 mg/mL.

8. The oral veterinary suspension of claim 7, wherein the suspending agent is present in an amount of about 5 mg/mL.

9. The oral veterinary suspension of claim 1, further comprising a sweetener.

10. The oral veterinary suspension of claim 9, wherein the sweetener is a natural sweetener, an artificial sweetener, or a combination thereof.

11. The oral veterinary suspension of claim 9, wherein the sweetener comprises sucrose, stevia, erythritol, xylitol, sucralose, saccharin, aspartame, or any combination thereof.

12. The oral veterinary suspension of claim 1, further comprising a preservative.

13. The oral veterinary suspension of claim 12, wherein the preservative comprises a sorbate, sorbic acid, an ascorbate, ascorbic acid, or any combination thereof.

14. The oral veterinary suspension of claim 1, further comprising one or more vitamins.

15. The oral veterinary suspension of claim 14, wherein the one or more vitamins comprise vitamin A acetate, vitamin B12, vitamin E acetate, or any combination thereof.

16. The oral veterinary suspension of claim 1, further comprising one or more nutritional minerals.

17. The oral veterinary suspension of claim 16, wherein the one or more nutritional minerals comprise sodium selenite.

18. The oral veterinary suspension of claim 1, wherein the oral veterinary suspension is stabile for at least one year.

19. The oral veterinary suspension of claim 1, wherein the iron salt has a particle size of about 25 μm to about 200 μm.

20. (canceled)

21. A method comprising:
generating an oral veterinary suspension comprising water, an iron salt that is iron fumarate, iron sulfate, or a combination thereof suspended in the water, and a suspending agent; and
delivering the oral veterinary suspension to a neonatal animal for prevention or treatment of an iron deficiency in the neonatal animal.
